(19) United States
(12) Patent Application Publication
(10) Pub. No.: US 2011/0195475 A1
(43) Pub. Date: Aug. 11, 2011

(54) ARTIFICIAL ENZYMES

(76) Inventors: Paul Bentley, Corrales, NM (US); Vincent Suzara, Albuquerque, NM (US)

(21) Appl. No.: 12/919,087

(22) PCT Filed: Feb. 28, 2009
(86) PCT No.: PCT/US09/35604

§ 371 Date: Feb. 14, 2011

Related U.S. Application Data
(60) Provisional application No. 61/032,118, filed on Feb. 28, 2008.

(51) Int. Cl. C12N 9/00; C08F 226/10; C08F 220/56; C07H 1/00; C08F 8/30

(52) U.S. Cl. 435/183; 526/264; 526/307.6; 527/300; 525/282

(57) ABSTRACT

The present invention is directed to the use of artificial polymers in the mimetization of enzymatic active sites and the carrying-out of catalysis using these artificial enzymes. Further, as used herein, an artificial enzyme refers more generally to a polymer-based scaffold for presenting specific chemically active atoms optimally for reactions, not just those that mimic natural enzymes. Various polymers can be used for this mimetization, including polyimides, polyurea, polyurethane, polyacrylic acid, and polylactic acid, as well as other polymers having properties and functionality that enable integration with natural and artificial amino acids, other molecules having nucleophilic and electrophilic groups (akin to the amine and carboxyl functionalities, respectively, of amino acids), as well as other molecules contributing unique chemical abilities not usually associated with the orthogonal functions inherent in most amino acids, i.e., amines, carboxyls, formamides, hydroxyls, mercaptyls and saturated hydrocarbons.

This application is related to U.S. provisional application 61/032,118, filed 28 Feb. 2008, incorporated herein by reference.

### TECHNICAL FIELD

This invention relates to the synthesis of enzymes and, in particular, to the synthesis of artificial enzymes comprising an organic polymer and an active site having biocatalytic functionality.

### BACKGROUND ART

Enzymes are large, conformationally complex structures made up of one or more chains of polypeptides which have been folded into specific shapes, the result of which is a biochemical catalyst. Living systems require the use of a wide range of small to medium size molecules for nutritive, structural and other purposes, and those smaller molecules are often only available as polymerized versions of larger molecules that must be broken down. For the generation of viable energy, polysaccharides, large proteins, and long chain fatty acids are the most common sources for carbon, other essential elements and reducing potential. Therefore, one of the most important classes of enzymes is that which performs catabolic functions, i.e., the breakdown of larger molecules into smaller units. Most of the catabolic enzymes which catalyze the breakdown of these large molecules are hydrolases, in that they promote the insertion of a water molecule between one or more monomers of a natural polymer. FIG. 1 shows a mechanism for depolymerization of peptidoglycan by carboxyl functions of a lysozyme. See D. J. Vocadlo et al., Nature 412, 835 (2001).

One of the many limitations to date in the development of artificial systems that accurately mimic biological processes is the development of mimetic enzymes that carry out functions analogous to naturally-occurring enzymes. A particular need is the mimetization of a subset of hydrolases known as glycoside hydrolases, enzymes that catalyze the breakdown of polysaccharides, or long chains of sugar-based monomers. Most of these enzymes perform at least some of their catalytic activities, i.e., binding, depolymerization, and release, in an active site or “catalytic cleft.” The latter term is used because of the conformation that the active site forms, as predicted by x-ray crystallography, nuclear magnetic resonance (NMR) and high speed computer models. In short, the active site appears to be some variation of a shape adequately described as an extended trough, mouth or open pocket. In some enzymes, the active site is completely enclosed, i.e., it takes the shape of a tunnel or invagination. In most cases, the region that contains the active site forms at least a semi-enclosed volume into which multi-saccharide substrates can, in sequence: (i) temporarily integrate, (ii) become exposed to catalytic amino acid residues and backbone structures, and then (iii) leave as products of the catalysis. In any case, the catalytic site or cleft defines a conformationally specific and chemically unique structure that, in the case of glycoside hydrolases, is well-suited to the breakdown of polysaccharides. FIGS. 2A and 2B show exemplary active sites. FIG. 2A shows a space filling model of hexokinase [Heriot-Watt University, Scotland]. FIG. 2B shows key residues and a metal ion cofactor (colored) in the active site of carboxypeptidase A [Dept. of Chemistry, Washington University].

Apart from the active site(s), the remainder of the enzyme is usually comprised of multiple structural subunits, which are often a multiplicity of structurally-folded linear chains of polypeptides. In most cases, this “non-catalytic” portion of the protein complex comprises over 90 percent of the total mass, number of amino acid residues, and volume taken-up by the complex. At first glance, this non-catalytic portion describes a surprisingly large proportion of protein dedicated to duties not directly related to its biological function, e.g., hydrolysis of polysaccharides. According to biochemical theories familiar to those practiced in the art, the vast majority of an enzyme serves as a structural scaffold, or support, in order to direct the smaller, key catalytic portions into a three-dimensional (3D) conformation that can undertake catalysis, or, more plainly stated, it serves to fold the catalytic portion of the enzyme into an active site. See, e.g., H. S. Taylor, Proc. R. Soc. (London) A108, 105 (1928); and Warshel and Levitt, J. Mol. Biol. 103, 227 (1976). This noted disproportion in relative residue commitment is not meant to diminish the role of the non-catalytic portion of the enzyme in supporting catalysis through exemplary functions, such as buffering the effects of substrate-induced shape changes, facilitating conformational changes that support catalysis, and serving as electron sources/sinks for the oxidation and/or reduction-based mechanisms that stabilize the transition states between reactants and products.

This a-priori imbalance of structural commitment also implies that the active site is composed of perhaps only 5 percent of an enzyme. For example, even in cellulases in the range of 1000 amino acid residues in size, catalysis could potentially take place in a volume defined by only approximately 50 amino acids. 3D modeling and other studies conclude that hydrolysis of even large polysaccharides, like cellulose, into smaller polysaccharides or monosaccharides can occur in the confined space defining the active site of a cellulase (e.g., an endo- or exo-glucanase, or beta-glucosidase) having a double-digit number of amino acids serving as catalyst-facilitators—mainly in transition state stabilization and hydrolysis—or in the direct support of such activity, e.g., binding, orientation, electron sources or sinks, buffering of redox potential, tribological support (i.e., facilitation of solvation) and release of product. FIG. 3A shows a topological representation of Cellulase 12A from Rhodothermus marinus, showing the substrate in yellow [Centre for Extremophile Research, University of Bath, UK]. FIG. 3B shows a stereo-representation of the active-centre loops of Cellulase 6B (red), Cellulase 6A-native (blue) and the Cellulase 6A glucose/cellotetraose complex (yellow). See G. J. Davies et al., Biochem. J. 348, 201 (2000).

Prior technologies to synthesize “plastic enzymes” or “synthetic enzymes/synzymes” include molecular imprinting, and those that include the integration of catalytically active proteins (in whole or in part) with polymers that act as structural scaffolds—often referred to as polymer-supported enzymes or matrix-immobilized enzymes.

Molecular imprinting is a technique for preparing polymeric materials that are capable of recognizing and binding a desired substrate, or template, with a high affinity and selectivity. Molecularly imprinted polymers (MIPs) have been used in many applications, including as stationary phases in chromatography and solid-phase extraction, as recognition elements in sensors and as catalysts of chemical reactions. In molecular imprinting, a template molecule is used to create a three-dimensional (3D) conformation on the polymer. Imprinting uses monomers whose positions are determined by their interaction with the template that are subsequently polymerized, thus approximately retaining the spatial relationship between the template and those key functional groups now incorporated in the polymer. To maintain a memory effect for the template molecule, the MIPs are typically highly cross-linked and rigid. The MIP thereby can rebind the template molecule or can mimic the active site of a catalyst that acts in a catalytic manner similar to the conformation-inducing template, or to a conformationally similar target molecule. The rationale behind this strategy is the “lock and key” model postulated by Fisher in the 1890s wherein the key is the template, the lock the catalytic site, and the polymer is made to mimic the parts of the lock that contact the key. See E. Fischer, Ber. Dtsch. Chem. Ges. 23, 799 (1890). It is widely known in the art that the drawbacks to MIPs are three-fold: (1) the dependence on the polymer to mimic a catalytic site, (2) the limitations of that polymer in maintaining what is presumed to be a biocatalytic conformation (and, by inference, the conformational flexibility required to maintain—at minimum—the initial substrate, transition state, and product molecules in a manner that transitions from substrate to product efficiently), and (3) the lack of functionalities that have been integrated into the polymers in order to adequately mimic the biocatalytic amino acid residues which perform the actual binding of target molecule, stabilization of transition state(s), and release of product. FIG. 4 shows an example of molecular imprinting of an acrylic-saccharide polymer. See Y. Kanekiyo et al., Chem. Commun., 2698 (2002).

More modern theories of how the conformation of an enzyme facilitates catalysis include the induced fit model postulated by Koshland. See D. E. Koshland, Proc. Natl. Acad. Sci. U.S.A. 44, 98 (1958). This model states that conformational variations in the protein-based polymer are necessary for, at minimum, recognition/binding, transition state stabilization, and release of product(s). The induced fit model infers that lock-and-key based catalytic polymers like MIPs have an inherent drawback due to their lack of flexibility to assume the correct range of conformational states. The model also implies that polymers which can mimic these conformational states, including catalytic oxidation/reduction facilitation and buffering, transition state stabilization, solvation, and other needed “active site” functionalities, would more closely resemble a biological enzyme as a system with catalytic capability.

Therefore, a need remains for an improved method to synthesize artificial enzymes that accurately mimic the flexible conformations and functions of naturally occurring enzymes. Specifically, these methods would modify artificial polymers with chemical functionalities and fold them into desired conformations, the result of which is a shaped polymer having the biocatalytic activity of enzymes.

### DISCLOSURE OF INVENTION

The present invention is directed to an artificial enzyme comprising a plastic or other organic molecules that are copolymerized to create an active site having biocatalytic functionality. The active site can comprise the aforementioned plastics and other polymerized organic molecules, natural or artificial amino acids, a molecule having nucleophilic and/or electrophilic groups, or molecules contributing unique chemical functions not usually associated with the orthogonal functions inherent in most amino acids. The unique chemical function can comprise keto-enol reactivity, ene-diol formation, Sn1 and Sn2 displacement, a diels-alder reaction, general metathesis, or a complex metallo-organic function, nitro aldol (Henry reaction), Knoevenagel reaction, Morita-Baylis-Hillman reaction, Steglich rearrangement, 1,3 dipolar cycloaddition, Strecker synthesis, allylation, alkylation, halogenation and amination. The plastic can comprise polyurea, polyimides, polyurethane, polyacrylic acid, or polylactic acid. The plastic can be co-polymerized with one or more other plastics, binding agents, or cross-linkers. The artificial enzyme can be a glycoside hydrolase.

The method enables the synthesis of artificial enzymes that accurately mimic the flexible conformations and functions of naturally occurring enzymes. These method can be used to modify artificial polymers with chemical functionalities and fold them into desired conformations, resulting in a shaped polymer having the biocatalytic activity of enzymes. Such shaped and functionalized polymers can also assume functionalities that biotic enzymes do not possess, i.e., perform trans-biotic catalysis, and can undertake such catalysis under conditions of solvation, temperature, pressure, electromagnetic radiation, and in the presence of inhibitory cofactors, which would normally neutralize the catalytic activity of biotic enzymes, i.e., under trans-biotic conditions. The nature of the artificial polymer in facilitating and supporting catalysis provides superior structural characteristics on the supported and functionalized active site, resulting in longer lasting and more readily usable catalytic systems, particularly for industrial processes.

### BRIEF DESCRIPTION OF THE DRAWINGS

The accompanying drawings, which are incorporated in and form part of the specification, illustrate the present invention and, together with the description, describe the invention. In the drawings, like elements are referred to by like numbers.

FIG. 1 shows a mechanism of depolymerization of peptidoglycan by carboxyl functions of a Lysozyme.

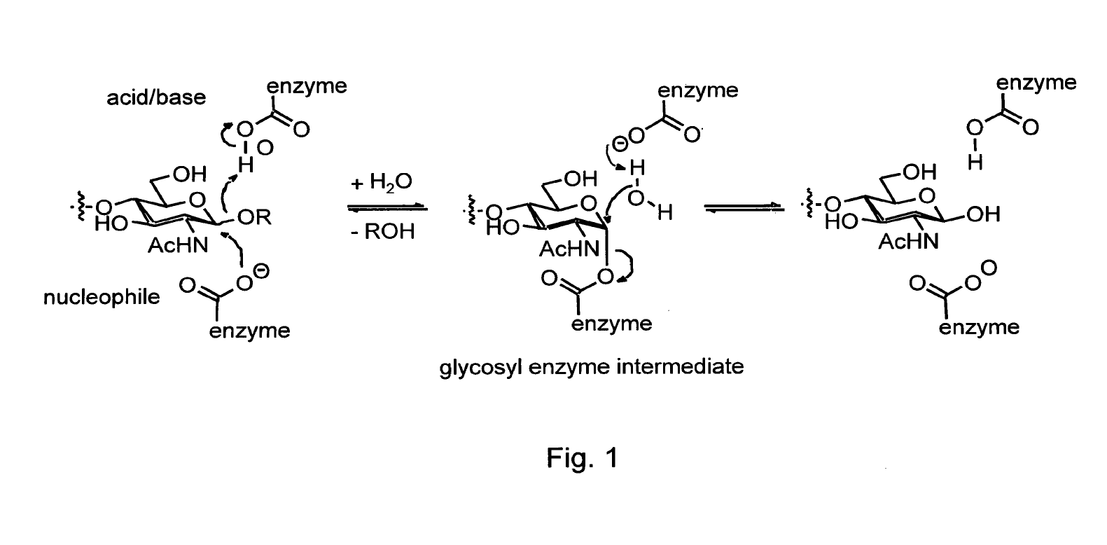

FIG. 2A shows a space filling model of Hexokinase. FIG. 2B shows key residues and a metal ion cofactor (colored) in the active site of carboxypeptidase A.

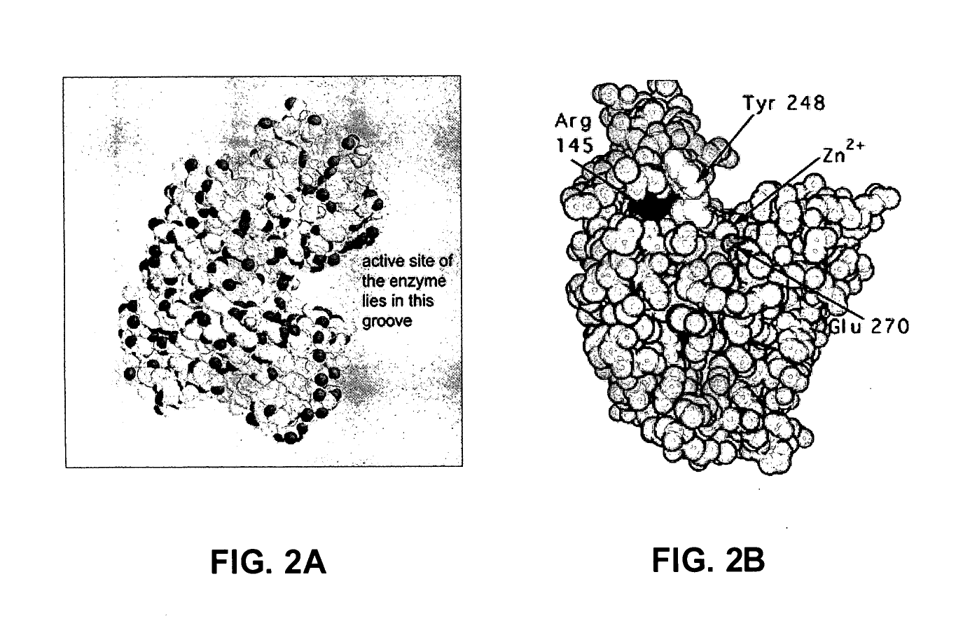

FIG. 3A shows a topological representation of Cellulase 12A from Rhodothermus marinus, showing the substrate in yellow. FIG. 3B shows a stereo-representation of the active-centre loops of Cellulase 6B (red), Cellulase 6A-native (blue) and the Cellulase 6A glucose/cellotetraose complex (yellow).

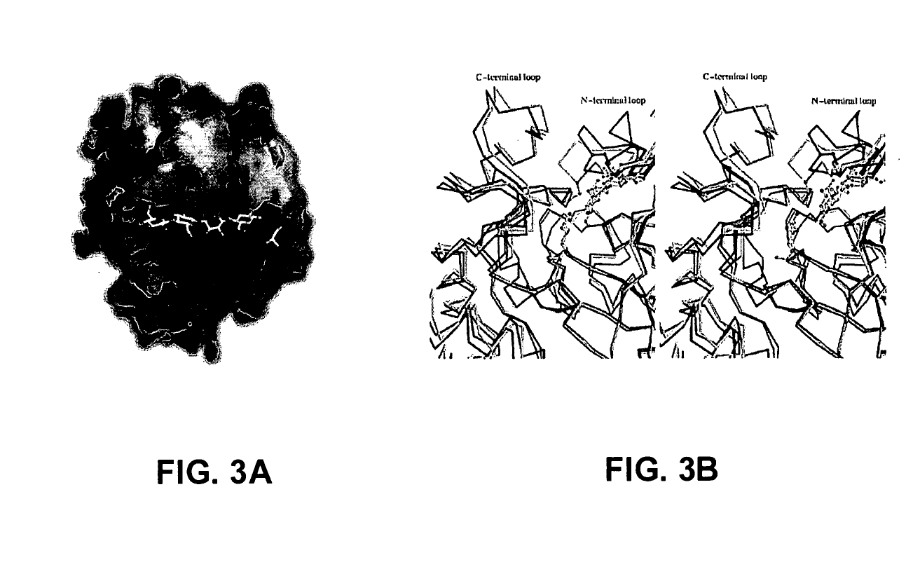

FIG. 4 shows an example of molecular imprinting of an acrylic-saccharide polymer.

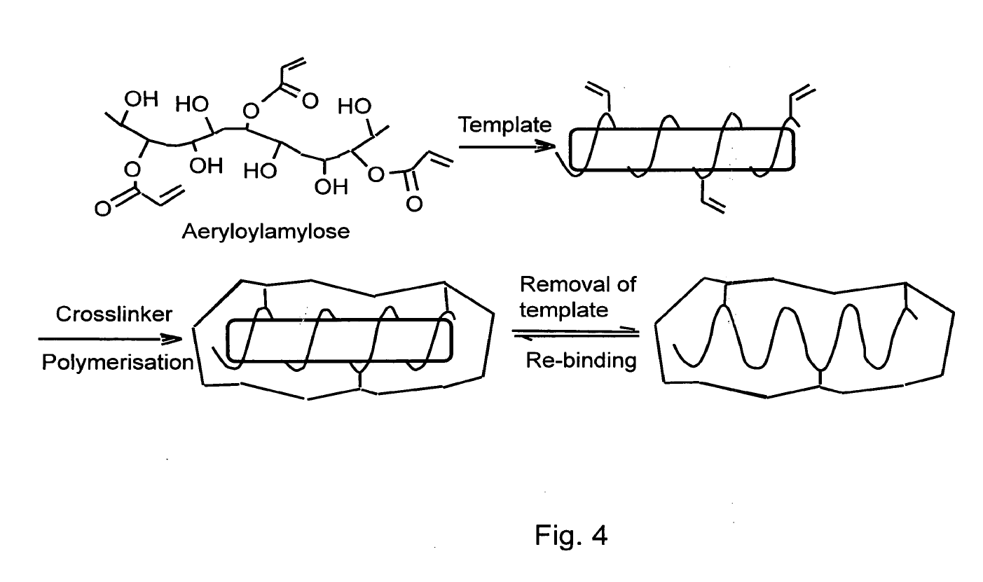

FIG. 5 shows types of polyacrylic acids having functionalities supportive of catalysis, copolymerization, folding and decoration with other monomers.

FIG. 6 shows a schematic illustration of an active site mimetic, with the artificial polymer (blue) supporting a catalytic site (green) wherein is localized a substrate (yellow with red and blue spheres.

FIG. 7 shows a folding tunnel representation of entropic states available to polypeptides, where “N”=the correctly folded state.

FIG. 8 shows a schematic illustration of the differences between in-line (left) and decoration (middle and right) modes of copolymerization.

FIG. 9 shows an example of a “classic” dual-monomer heterogeneous copolymer.

FIG. 10 shows an exemplary synthetic method comprising backbone/in-line copolymerization of PEGylated Leucines.

FIG. 11 shows an exemplary synthetic method comprising unnatural amine-acids that induce folds/direction changes in an in-line copolymerization scheme.

FIG. 12 shows an exemplary synthetic method comprising polypeptide-based “conformamers” that can be used as scaffolds in a decoration copolymerization scheme, likely with N′-functionalized monomers, resultant tertiary amides.

FIG. 13 shows an exemplary synthetic method comprising plastic-based “conformamers” that can be used as scaffolds in a decoration copolymerization scheme, likely with functionalized monomers to attach amino acids, etc.

FIG. 14 shows (top) an exemplary synthetic method comprising plastic-based scaffold for heterogeneous copolymerization with single stand DNA to resultant prefolded addressable template, (middle) types of reactions possible with reagents templated to proximity via DNA hybridization, and (bottom) unnatural amino acids that may be used in polymerization and/or orthogonal functionalization.

FIG. 15 shows an exemplary synthetic method that uses aldehyde functionality to induce backbone folds, cross-link, catalyze and serve as functionalization points for other monomers.

FIG. 16 shows exemplary plastic-based polymers.

FIG. 17 shows a concept of small, sub-molecular foldamers.

FIG. 18 shows a functionalization with organometallics.

FIG. 19 shows the functionalization of a polystyrene terminus with maleimide for binding of mercaptyl-containing groups, e.g., Cysteine.

FIG. 20 shows an exemplary sub-molecular unit folded into a “cleft” conformation, and pre-functionalized with amine and hydroxyl groups [figure taken from a monomer of silica, Prof. Q. Yang, Acad. Sinica, PRC]

FIG. 21 shows an exemplary sub-molecular unit folded into a cleft conformation, and pre-functionalized with (from counterclockwise) amine, carboxyl acid, aldehyde, hydroxyl, imidazyl and pyridyl moieties, with each functionalization localized on an “address” unique to each phenyl group-based monomer of the structure. The unit is anchored to a solid phase, shown by the thick and angled lines at the bottom. See G. C. Lloyd-Jones, Annu. Rep. Prog. Chem. 97 (2001).

FIG. 22 shows an exemplary sub-molecular unit composed of multiple bi-phenyl ring monomers polymerized into a five address cleft, each with orthogonal chemical function potential. This structure is also solid-phase anchored via polymerized and cross-linked groups shown at the bottom.

FIG. 23 shows an exemplary sub-molecular unit folded into a truncated ring conformation with functionalizable “addresses” shown by the ten (10) numbered, large single or double-spherical moieties on the inner portion of the ring. See U.S. Pat. No. 6,716,370 to Kendig.

FIG. 24 shows an exemplary top-on view of five (5) sub-molecular units cross-linked into a supra-molecular structure, creating a catalytic cleft of progressively increasing cleft enclosure size—from bottom to top. Each unit can be orthogonally functionalized as described in FIGS. 21-23.

FIG. 25 shows an off angle side view of an exemplary idealized product of the catalytic cleft geometry, composed of a multiplicity of truncated ring shaped, and inner-surface functionalized, sub-molecular units that have been cross-linked into a supra-molecular structure of progressively increasing enclosure size—from right to left. Also shown is a circular ring “anchor,” or seed shape, on the extremity that is used as a polymerization guide for iterative addition of truncated ring units, to create and preserve the overall “cleft” conformation of the product. A short, 30 glucose monomer-long cellulose molecule is shown above for size comparison.

### BEST MODES AND INDUSTRIAL APPLICATION OF THE INVENTION

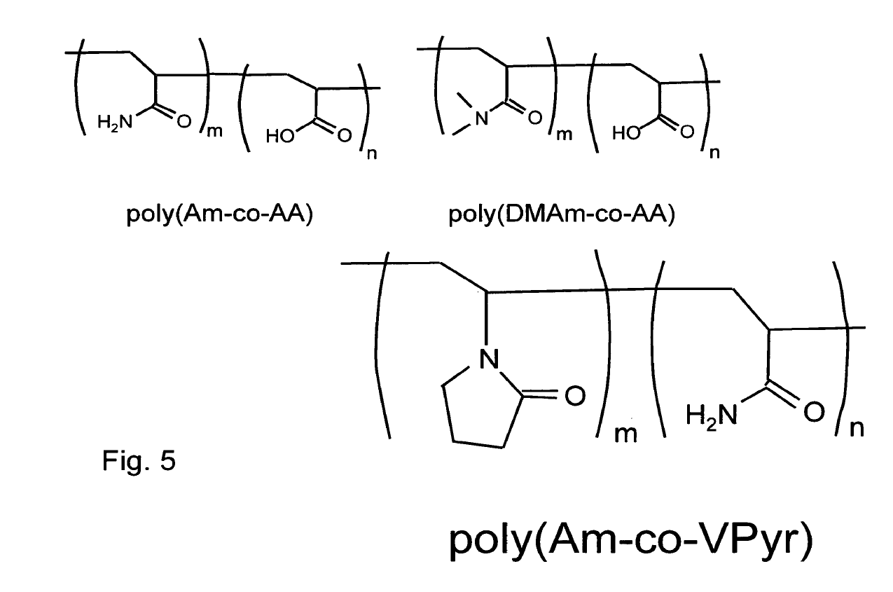

The present invention is directed to the use of artificial polymers in the mimetization of enzymatic active sites and the carrying-out of catalysis using these artificial enzymes. Further, as used herein, an artificial enzyme refers more generally to a polymer-based scaffold for presenting specific chemically active atoms optimally for reactions, not just those that mimic natural enzymes. Various polymers can be used for this mimetization, including polyimides, polyurea, polyurethane, polyacrylic acid, and polylactic acid, as well as other polymers having properties and functionality that enable integration with natural and artificial amino acids, other molecules having nucleophilic and electrophilic groups (akin to the amine and carboxyl functionalities, respectively, of amino acids), as well as other molecules contributing unique chemical abilities not usually associated with the orthogonal functions inherent in most amino acids, i.e., amines, carboxyls, formamides, hydroxyls, mercaptyls and saturated hydrocarbons. These “trans-amino acid” functions can enable keto-enol reactivity, ene-diol formation, Sn1 and Sn2 displacements based on halides, diels-alder reactions, general metathesis reactions, complex metallo-organic functions, metal chelating capacity, Henry reaction, Knoevenagel reaction, Morita-Baylis-Hillman reaction, Steglich rearrangement, 1,3 dipolar cycloaddition, Strecker synthesis, allylation, alkylation, halogenation and amination, and other capabilities provided by the integration into the backbone polymer of groups not limited to the twenty naturally-occurring amino acids. FIG. 5 shows types of polyacrylic acids having functionalities that can support catalysis, copolymerization, folding and decoration with other monomers [Univ. of Concepcion, Chile].

The active site of most enzymes, as is known in the art, is a structural region defining a sub-portion of the overall protein-based complex. The active site facilitates an increased rate of conversion of starting material to product, i.e., catalysis, by mechanisms which can be lowering of the activation energy, stabilization of transition states between substrates and products, contribution of chemical functions, and stabilizing geometry. An enzyme's active, or catalytic, site comprises a unique assembly of amino acid residues arranged in a particular 3D conformation which is highly specific for recognizing and modifying a target molecule, or substrate, (or an inducer or repressor which mimics the substrate) using the summation of the orthogonal functions of the residues, their locations in 3D space, interfacial solvation, and the conformational flexibility of the active site as facilitated by the scaffold of the larger protein-based enzyme complex.

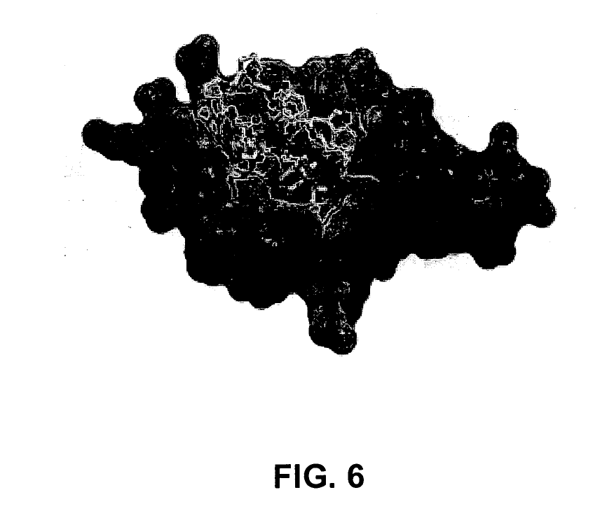

An improved, artificial polymer-based sofa natural enzyme would accomplish the same biocatalytic functions as the natural enzyme in a manner that improves upon the biocatalytic function or functions accomplished and the structure of the overall molecule. With regard to improvements on the structure and/or functions of the active site per se, this may include superior characteristics based on: (i) increased rates of catalysis based on entropic and enthalpic modulations of transition states and leaving groups, (ii) decreased numbers of amino acid and other orthogonal functionalities and residues required to accomplish certain catalyses, (iii) a wider spectrum of available chemistries (and, thus, potential catalyses that can be performed) based on the inclusion of chemical functionalities not provided by naturally occurring amino acids, (iv) a broadened range of substrates that can be catalyzed, and (v) increased control over the conformation of the active site, and, thus, of the sequential steps of catalysis, via replacement, in whole or in part, of the non-catalytic portion of the mimetic enzyme with artificial polymers which may include the aforementioned plastic-based polymers. FIG. 6 shows a schematic illustration of an active-site mimetic, with the artificial polymer (blue) supporting a catalytic site (green) wherein is localized a substrate (yellow with red and blue spheres). [Robinson Group, Organic Chemistry Institute, University of Zurich, CH].

In general, these polymers (referred to herein as “plastics” to describe a generalized group of organic polymers, the monomers of which have functionalizations which enable their polymerization via condensation, free radical propagation, dehydration and other means) may be prepared alone or co-polymerized with one or more other monomers/oligomers/polymers, chemical functionalities with the capacity to bond other chemicals, or cross-linkers and solvents, in a variety of different shapes and sizes. More dutiful and iterative polymerization of plastic monomers, binders, etc., can also result in polymeric products that can conform to a wide range of geometric structures, e.g., dendrimers, well-defined spheres, fractal-patterned 3D nets, block or layered copolymers (in which plastics sequester according to design in the course of polymerization from the liquid or colloidal to the solid phase) arrayed and parallel sheets, and helices. Under controlled conditions, and with diligent use of monomers and other starting material, the plastic polymers may assume shapes that closely mimic natural enzyme active sites.

In addition to assuming an active site-like conformation, the plastic can be co-polymerized with amino acids directly into its carbon backbone, or the backbone can be “decorated” with amino acids in a manner that does not significantly affect the ability of the polymer to fold as intended. Below are described exemplary methods for the construction of plastic polymer-based active site mimetics that assume structures roughly described as clefts or troughs, similar to the catalytic regions of glycoside hydrolases.

As well as assuming a desired shape, and including amino acids or other active monomers or functionalities into this system, it is important that the resultant polymer mimic the flexibility of active sites. Many ‘shape memory’ polymers are based on plastics (e.g., one of the above listed, or others) which are designed to assume a restricted set of conformational possibilities, i.e., a range of conformations, and maintain the range even after undergoing conformations which take the polymer out of its designed range. This is important in the mimetization of an active site, since no enzymes have a completely static catalytic center, and all glycoside hydrolases act by the expressed conformational flexibility to bind, adhere, change conformation around, cleave, stabilize and release a smaller chain of sugars than the one it started out with. A mimetic polymer preferably folds like a natural active site, has the same amino acids as an active site (or have groups that carry out the same or better chemical functions as those residues), and is also able to flex and change shape like an active site. All the while, the mimetic polymer must keep to a certain restricted set of conformations, i.e., not be “too flexible,” so as not to risk potentially unraveling and losing its shape memory.

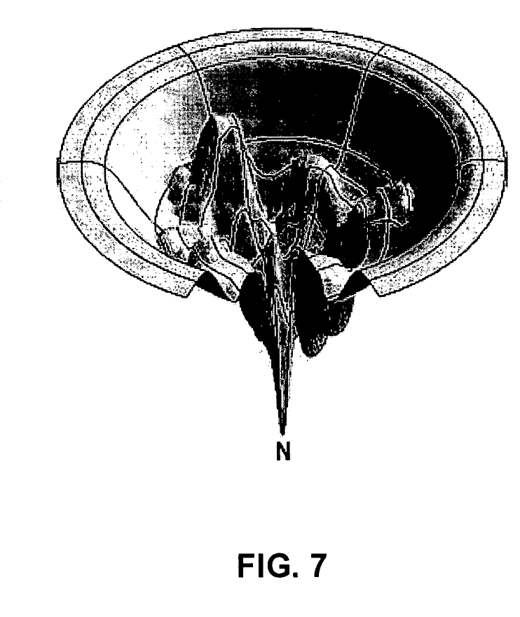

One of the difficulties in converting polypeptides into usable enzymes, i.e., into proteins with catalytic functions, as currently understood to practitioners of the art, is the uncertainty inherent in folding a linear, native sequence which has been synthesized as such or one that has been completely denatured into the native form. Technology does not currently exist for the reliable and predictable folding of linear peptides larger than around 50 amino acids in length into a limited repertoire of shapes. This is a primary reason why there are few commercially-available, artificially synthesized polypeptides larger than about fifty (50) residues in length that claim any complex biological activity, i.e., that are “true enzymes.” According to energy landscape theory, the final conformation of an enzyme is a summation of progressively lower free energy states arrived at after conformational transitions within an energy based “folding tunnel,” which had been postulated two years earlier. See Gulukota and Wolynes, Proc Natl Acad Sci USA. 91, 9292 (1994) and Leopold et al., Proc Natl Acad Sci USA. 89, 8721 (1992). FIG. 7 shows a folding tunnel representation of entropic states available to polypeptides, where “N”=the correctly folded state [Dept. of Biochemistry, Univ. of Toronto, CAN]. Though the final conformation is energetically ideal in this respect, particularly with regard to minimized entropy, both theory and experiment show that many metastable states exist that the “pre-enzyme” may assume due to: (1) those states having relatively low, though not minimal, free energy levels, and (2) the positive energy investment that must be made to unfold the pre-enzyme over the transitional energy barrier before it can be correctly folded again. In short, a high probability exists for the pre-enzyme to become recalcitrant in one or more of those incorrect states and remain catalytically inactive. In the current art, the folding of any polypeptide over a 50-mer size requires teraflop supercomputers to predict and presents many possible metastable “pitfalls” into which the 50-plus-mer may fall into. Consequently, no method exists to reliably and correctly fold enough of the molecules to challenge the production of enzymes in genetically engineered microorganisms.

The present invention avoids the folding issue entirely by directly creating active sites using plastics or other appropriate polymers to facilitate specific catalytic functions within a defined geometric range of conformations via scaffolding or other support. The folded conformation of a natural enzyme is used merely as inspiration for the shape, chemical character (i.e., functions based on amino acid or other monomeric residues), and acceptable range of conformations of the active sites to be mimetized by plastic-based polymers and other reagents. Exemplary structures that can be constructed include troughs and clefts that mimic the shape of glycoside hydrolase-to-polysaccharide recognition, binding, transition state stabilization, depolymerization, buffering and release sites. Exemplary functionalities include the amino acid residues in those active sites. Conformations and ranges therein can be designed into the plastic polymer backbone by supercomputer models, NMR and X-ray crystallography.

Polymers, in general, can support the function of known catalytically-active enzymes by strengthening the protein from the outside of the molecule and, in some instances, replace one or more amino acid residues resulting in a co-polymerized protein-plastic mimetic. One example of the latter is the use of polyethylene glycol/polyethylene oxide (PEG/PEO) chimaeric systems that directly attach enzymatic proteins to a solid phase by PEGylation of one or more residues to the extended colloid, which may itself be covalently bonded to a solid core of polystyrene (PS) or other amenable resin. In this “ball and stick” strategy, the PEG molecules form the extended support while the enzymes are folded into active conformations at the termini. Another strategy is the use of polymers as matrix supports in processes wherein it is desirable for the enzyme to be maintained in the solid phase, yet not be integrated as intimately with the polymer as in the PEG example above. A common strategy of functionalizing an enzyme for matrix support is the orthogonal modification or functionalization of one or more peripheral residues, relatively distal from the binding or catalytic sites, for subsequent inclusion to a solid phase matrix. Examples of such are biotinylation for binding to streptavidin on the solid phase, binding of Lysyl or Arginyl residues to N-hydroxysuccinimide on the matrix, and binding of free Cystl residues to maleimide residues on solid phase.

### Example Embodiments and Applications

Exemplary monomers that can be used to form copolymerized artificial enzymes include i) naturally occurring alpha amino acids; ii) artificial alpha, beta-, gamma- or other extended backbone amino acids; iii) N′-functionalized amino acids of various backbone lengths; iv) other monomers having functionalities that facilitate their inclusion into the polymer. Such functionalities, if inspired by amino acids, can include nucleophilic groups (e.g., primary or secondary amines, hydroxyl, mercaptyl and phosphate groups) usefully distal to electrophilic groups (e.g., carboxylic acids and unsaturated carbons, i.e., alkenes and alkynes), such that the orthogonal chemical function on the monomer presents an orientation of that functional group in the overall polymer useful for catalysis; and v) plastic-based monomers that, in addition to their resultant and desired roles of forming part of the supra-molecular backbone or backbones, are (i) orthogonally functionalized with chemical functions that contribute to catalysis, or (ii) orthogonally functionalized to accept an amino acid, DNA-based nucleotide, or other catalytically contributive monomer, and oligomers thereof, in a “decoration” mode (described in additional detail below) whereby the orthogonally active monomer does not significantly contribute to the overall shape or 3D conformation of the supra-molecular structure.

An exemplary artificial enzyme comprises the copolymerization of plastic-based monomers with other monomers having orthogonal functionality that contribute to catalysis in a manner that utilizes the non-orthogonal portion of the latter as a subunit of the resultant molecule's primary backbone. It is understood in the art that “in-line” or “backbone” copolymerization, in the sense described herein, and as will be used conceptually henceforth, describes the inclusion of a plurality of monomer families into the solid phase such that each unique family of monomers integrates into the resultant supra-molecular assembly on an equal basis—with regard to the degree of contribution to the overall shape, folding or 3D conformation of the supra-molecular assembly—to the other unique monomer families. This type of polymerization has also been described as “selective chain growth”. See C. J. Hawker and K. L. Wooley, Science 309, 1200 (2005).

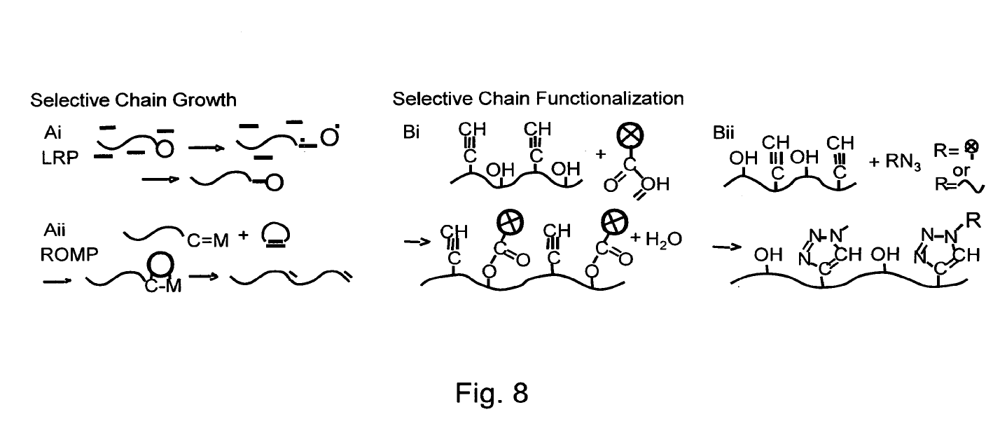

Another exemplary artificial enzyme comprises plastic-based monomers and others having orthogonal functionality that contribute to catalysis, copolymerized in manner that does not utilize the non-orthogonal portion of the latter as a subunit of the resultant primary backbone. It is understood in the art that “side group” or “decoration” copolymerization, in the sense described herein, and as will be used henceforth, describes the inclusion of a plurality of monomer families into the product such that each family therein contributing orthogonal functions integrates into the supra-molecular assembly on an unequal basis—with regard to 3D conformation—to the primary monomer families that form the actual and understood scaffold. This manner of polymerization has also been described as “selective chain functionalization”. FIG. 8 shows schematic illustrations of the differences between in-line (left) and decoration (middle and right) modes of copolymerization. See Hawker and Wooley.

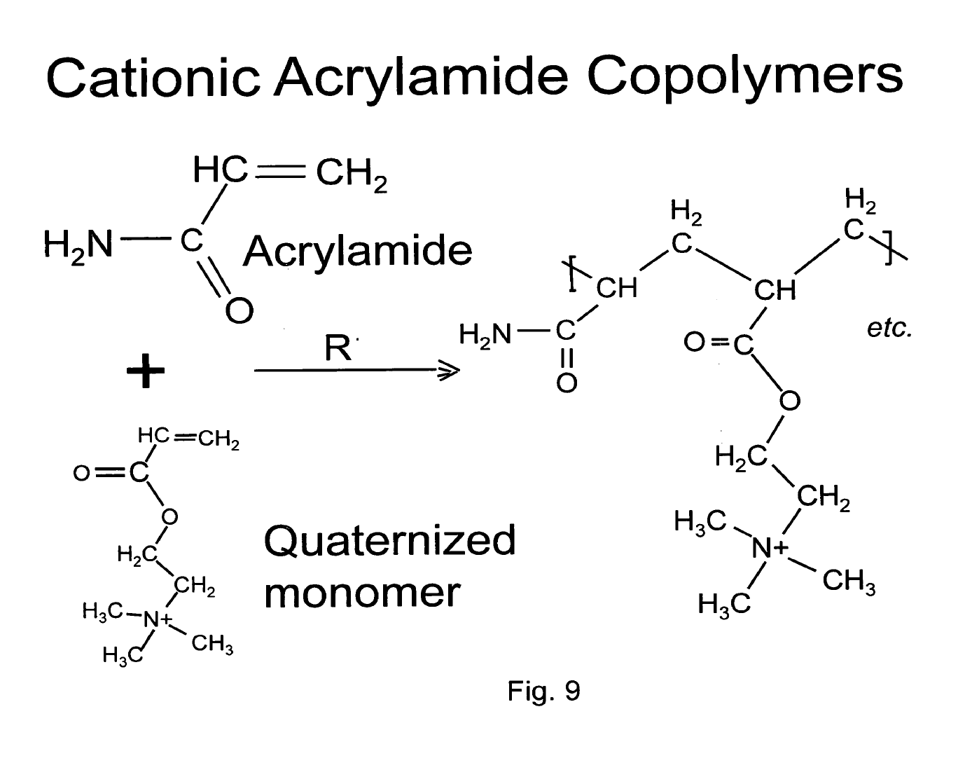

Another exemplary artificial enzyme comprises the copolymerization of plastic-based monomers with other monomers having orthogonal functionality that contribute to catalysis, in manner that utilizes the non-orthogonal portion of the latter as a secondary backbone relative to the primary backbone represented by the polymerized plastic. It is understood in the art that this “mated” or “classic” copolymerization, in the sense described herein, and as will be used henceforth, describes the inclusion of a plurality of orthogonally-functional monomer families such that the latter integrates into the resultant molecule on either an equal or unequal basis, with regard to 3D conformation, to the other unique monomer families. The contribution of the functional monomers relative to the understood “primary backbone” of polymerized plastic can be globally equal, globally unequal, or in variations thereof at each residual location with regard to overall contribution to 3D conformation of the complex. It is understood in the art that the heterogeneous nature of the mated and copolymerized monomers results in a supra-molecular assembly having folds, shapes and a 3D conformation that is unique from the polymerization of the mated monomers alone. FIG. 9 shows an example of a “classic” dual-monomer heterogeneous copolymer [Illustration from Prof. Martin Hubbe, North Carolina St. Univ.].

Another exemplary artificial enzyme comprises heterogeneous copolymerization of plastic-based monomers with oligopeptides to make a polymer supported active-site mainly mimetic. This structure would involve a decoration-type copolymerization scheme wherein nucleophilic and electrophilic functions would exist on the main polymer backbone, spatially directed and concordant with the locations of carbonyl and secondary amide groups on polymerized alpha amino acids, to form a block copolymer of plastic and a polypeptide.

Another exemplary artificial enzyme comprises heterogeneous copolymerization of plastic-based monomers with single strand DNA to make an addressable template for oligonucleotides backbone-functionalized with functional groups pertinent to catalysis reactivity, or recognition of amino acids, etc., and also functional groups inert to catalysis, reactivity, or recognition. This structure would also involve a decoration-type copolymerization scheme wherein functions would exist on the main polymer backbone, spatially directed and concordant with the locations of phosphate groups on polymerized nucleotide monophosphates (like single stranded DNA or RNA), to form a block copolymer of plastic and a nucleic acid. The latter may be 5′-phosphate modified with additional functions to enable this form of copolymerization, e.g., the creation of 5′-phosphoramidate, 5′-phosphorothioate, and 5′-phosphohydrazide groups reactive to concordant functions on the plastic polymer backbone.

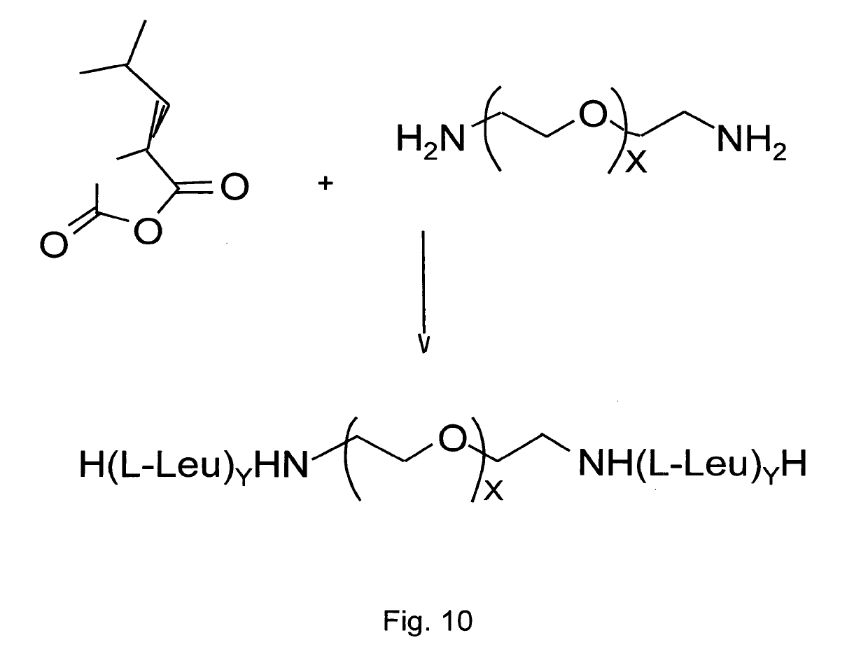

FIG. 10 shows an exemplary synthetic method comprising backbone/in-line copolymerization of PEGylated Leucines. See R. W. Flood et al., Org. Lett. 3, 683 (2001).

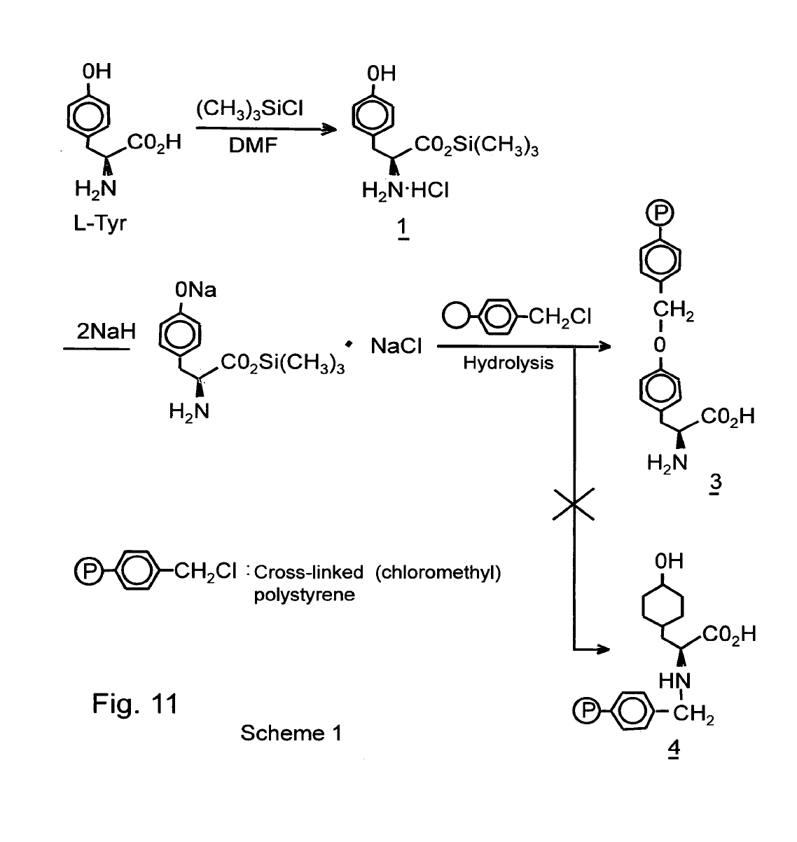

FIG. 11 shows an exemplary synthetic method comprising unnatural amine-acids that induce folds/direction changes in an in-line copolymerization scheme. See S. Itsuno et al., Polymer Bulletin 20, 435 (1988).

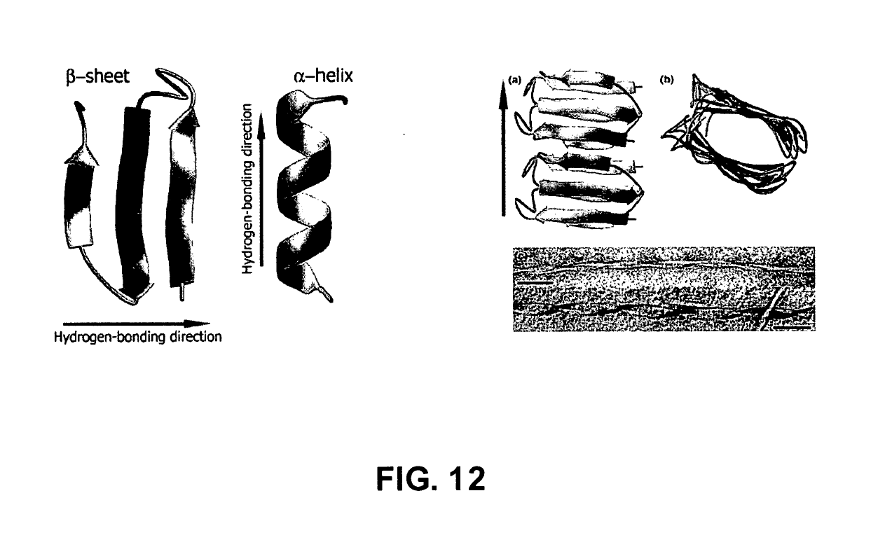

FIG. 12 shows an exemplary synthetic method comprising polypeptide-based supra-molecular “conformamers” that can be used as scaffolds in a decoration copolymerization scheme, likely with N′-functionalized monomers, resultant tertiary amides. See C. E. MacPhee and D. N. Woolfson, Curr. Ooin. Solid State and Matls. Sci. 8, 141 (2004).

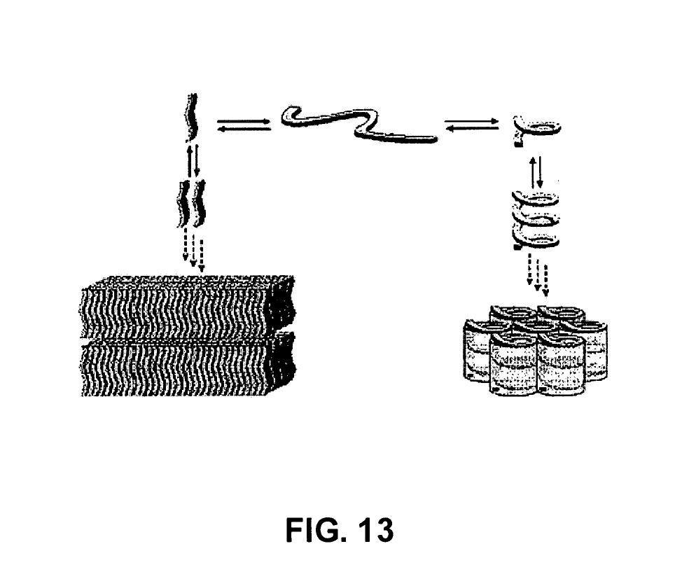

FIG. 13 shows an exemplary synthetic method comprising plastic-based “conformamers” that can be used as scaffolds in a decoration copolymerization scheme, likely with functionalized monomers to attach amino acids, etc. See K. L. Wooley et al., PNAS 97, 11147 (2000).

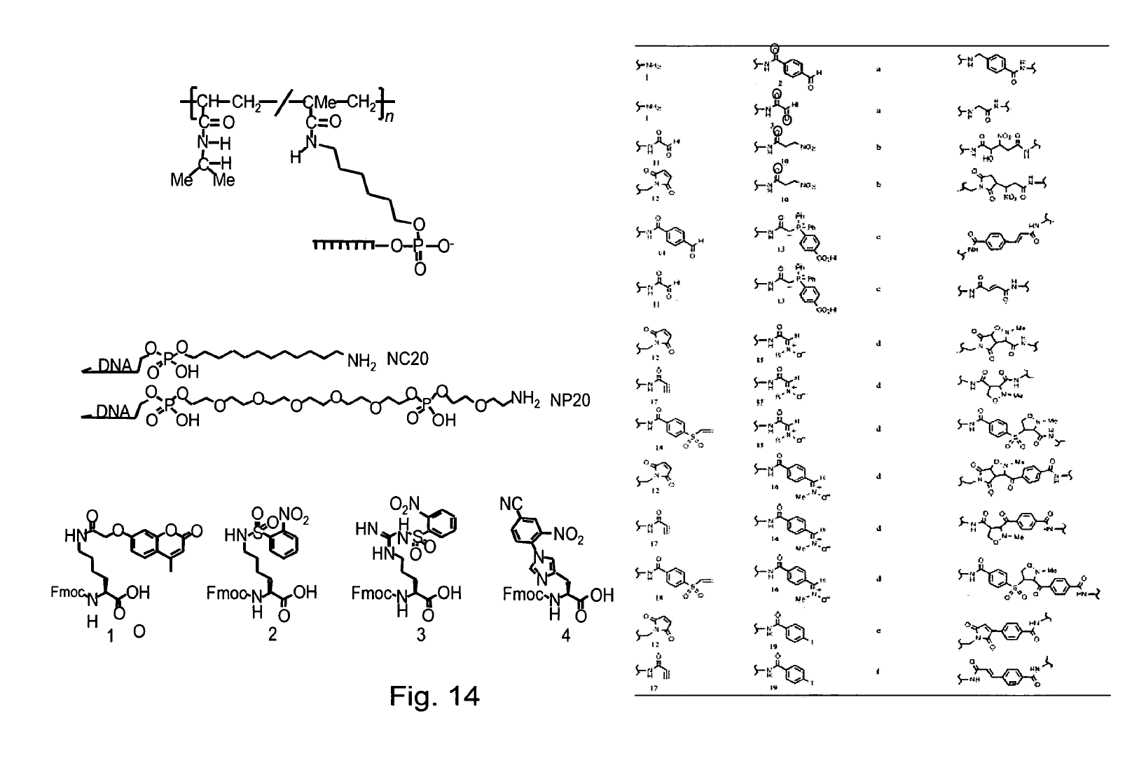

FIG. 14 shows (top) an exemplary synthetic method comprising a plastic-based scaffold for heterogeneous copolymerization with single stand DNA to resultant prefolded addressable template, (middle) types of reactions possible with reagents template to proximity via DNA hybridization, and (bottom) unnatural amino acids that can be used in polymerization and/or orthogonal functionalization. See D. Umeno et al., Chem. Commun., 1433 (1998); K. J. Gartner et al., Angew. Chem. Int. Ed. 41, 1796 (2002); and D. R. Halpin et al., PLOS Biology 2, 1031 (2004).

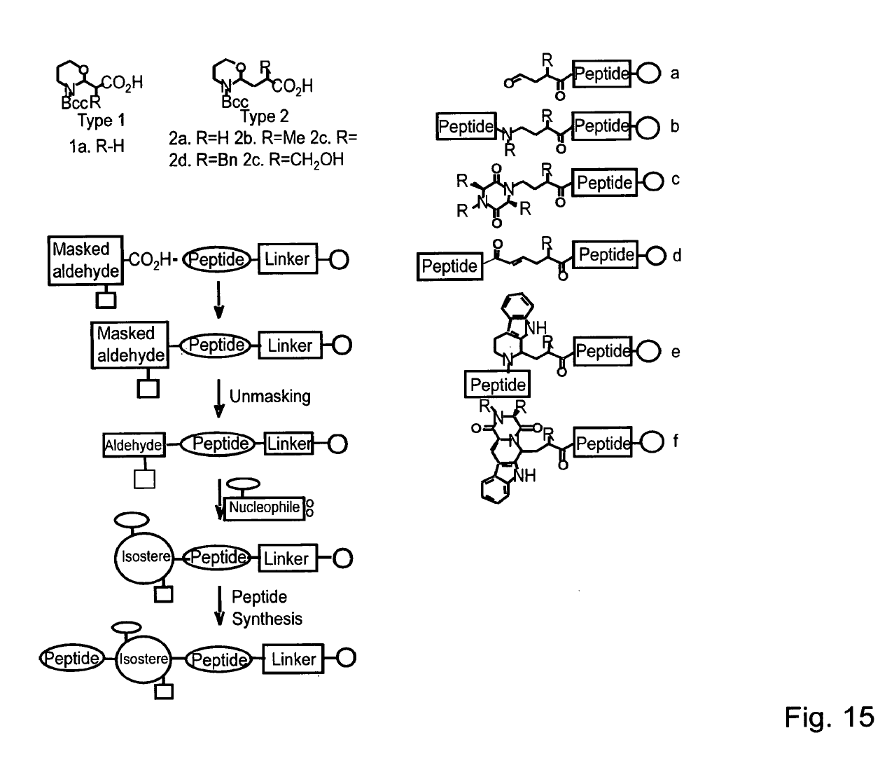

FIG. 15 shows an exemplary synthetic method that uses aldehyde functionality to induce backbone folds, cross-link, catalyze and serve as functionalization points for other monomers. See T. Groth and I. M. Melda, Comb. Chem. 3, 45 (2001).

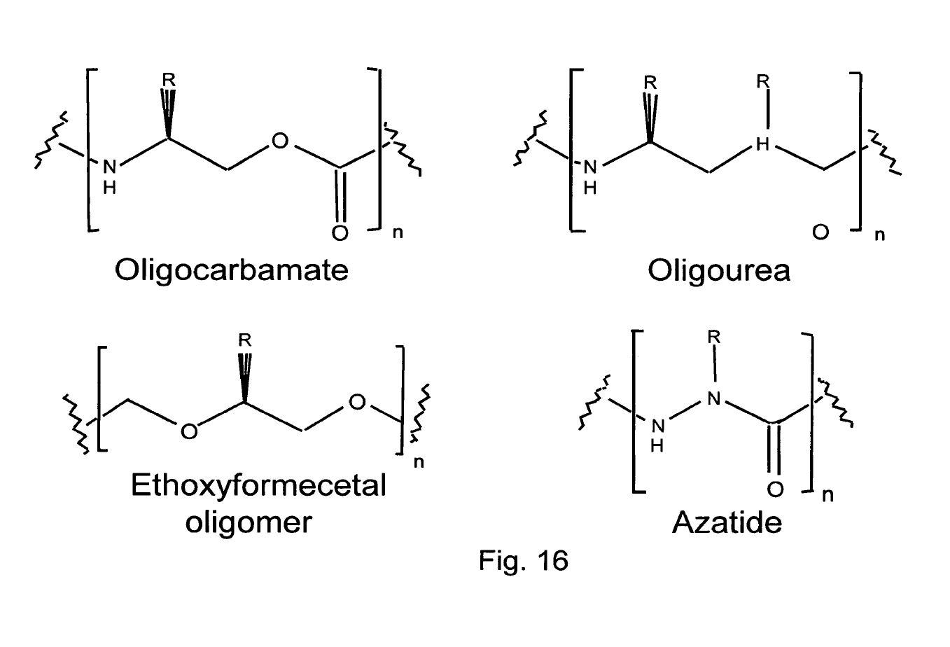

FIG. 16 shows an exemplary plastic-based polymer. See A. E. Barron and R. N. Zuckerman, Curr. Ooin. Chem. Biol. 3, 681 (1999).

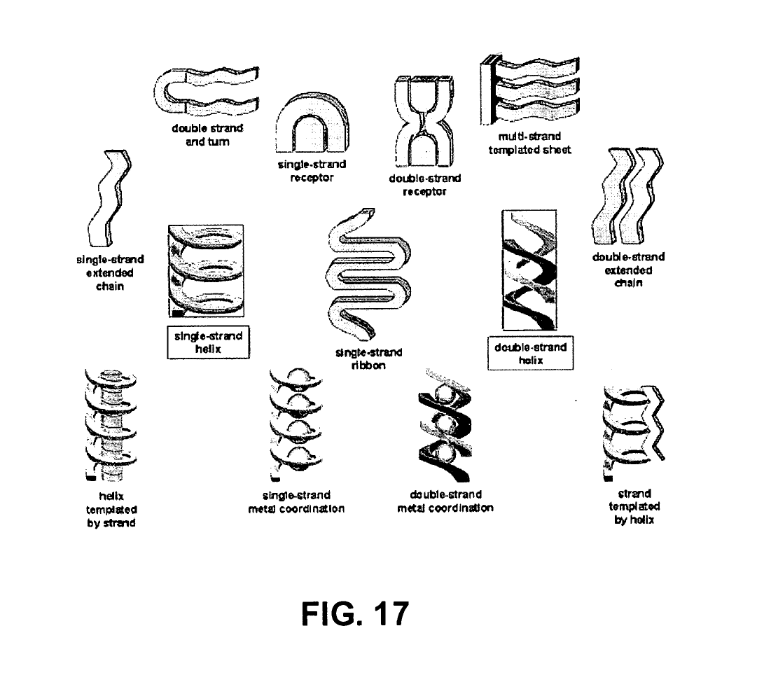

FIG. 17 shows an example of the concept of small, sub-molecular foldamers. See D. J. Hill et al., Chem. Rev. 101, 3893 (2001).

An example of the concept of active site flexibility has been described by Tsou. See C. L. Tsou, Anal. NY. Acad. Sci. (2002).

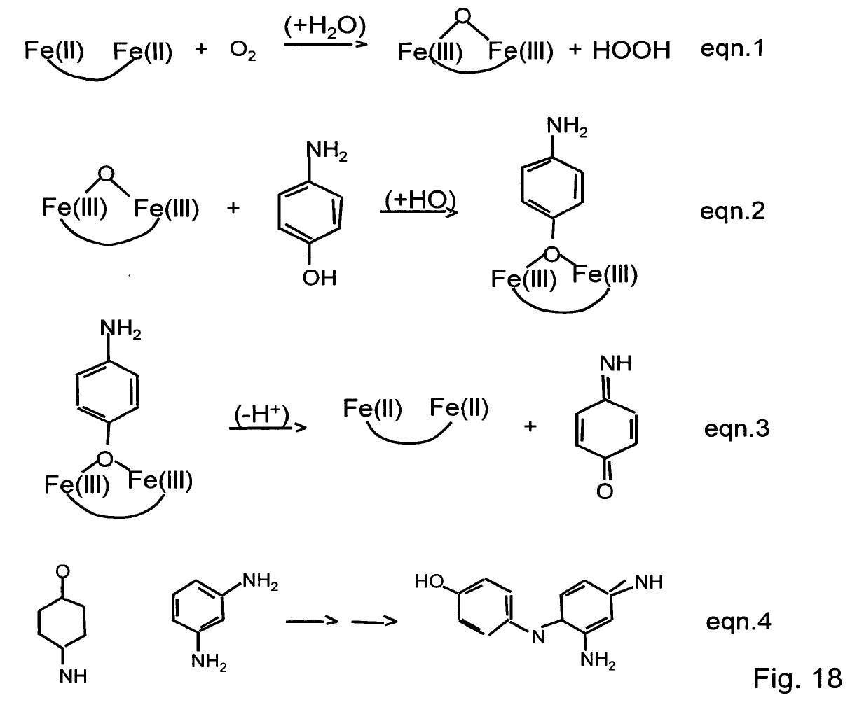

FIG. 18 shows an example of functionalization with organometallics. See J. Kaplan and W. F. Degrado, PNAS 101, 11566 (2004).

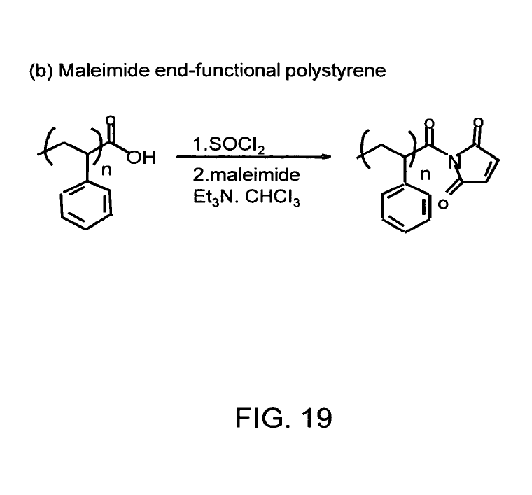

FIG. 19 shows the functionalization of a polystyrene terminus with malemide for binding of mercaptyl-containing groups, e.g., cysteine.

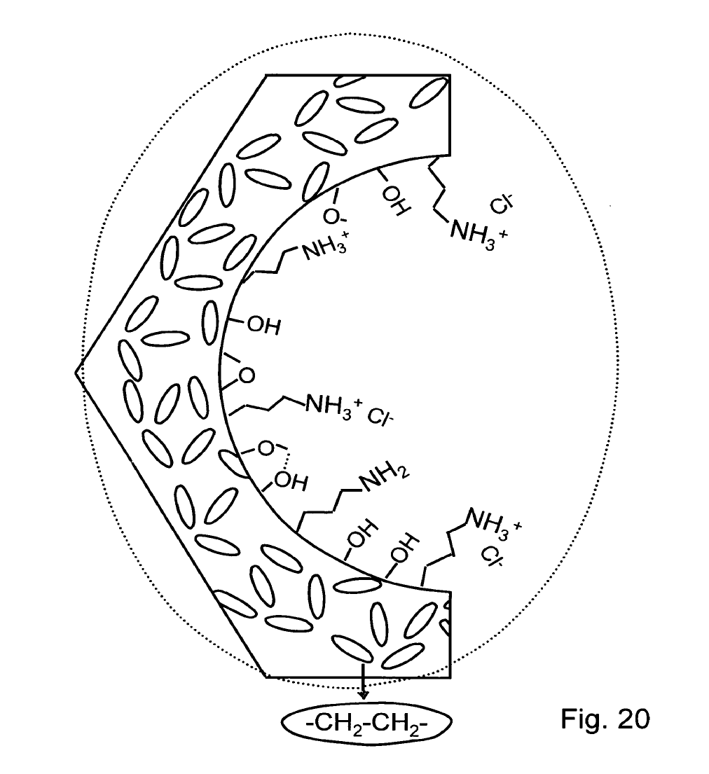

FIG. 20 shows an exemplary sub-molecular unit folded into a “cleft” conformation, and pre-functionalized with amine and hydroxyl groups [figure taken from a monomer of silica [Prof. Q. Yang, Acad. Sinica, PRC].

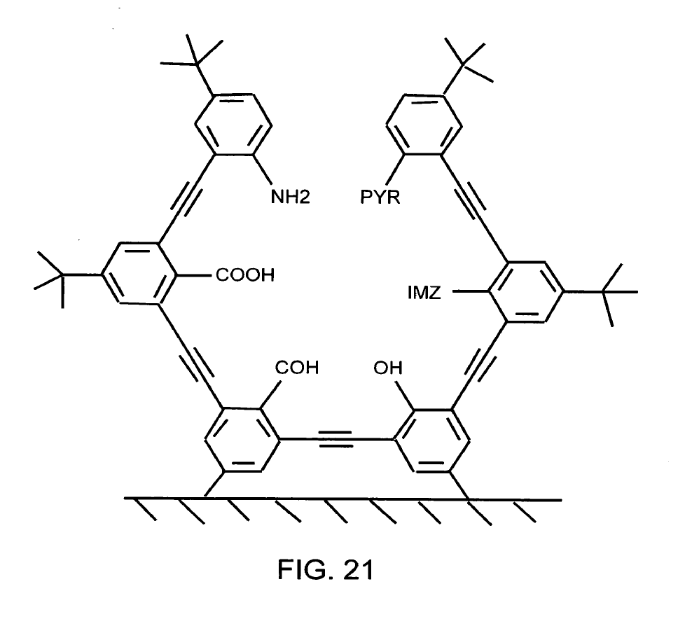

FIG. 21 shows an exemplary sub-molecular unit folded into a cleft conformation, and pre-functionalized with (from counterclockwise) amine, carboxyl acid, aldehyde, hydroxyl, imidazyl and pyridyl moieties, with each functionalization localized on an “address” unqiue to each phenyl group-based monomer of the structure. The unit is anchored to a solid phase, shown by the thick and angled lines at the bottom. See G. C. Lloyd-Jones, Annu. Rep. Prog. Chem. 97 (2001).

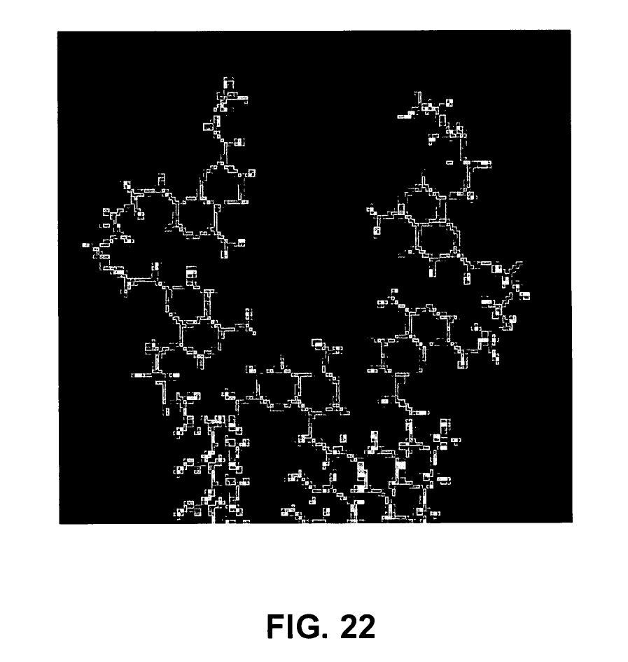

FIG. 22 shows an exemplary sub-molecular unit composed of multiple bi-phenyl ring monomers polymerized into a five address cleft, each with orthogonal chemical function potential. This structure is also solid-phase anchored via polymerized and cross-linked groups shown at the bottom.

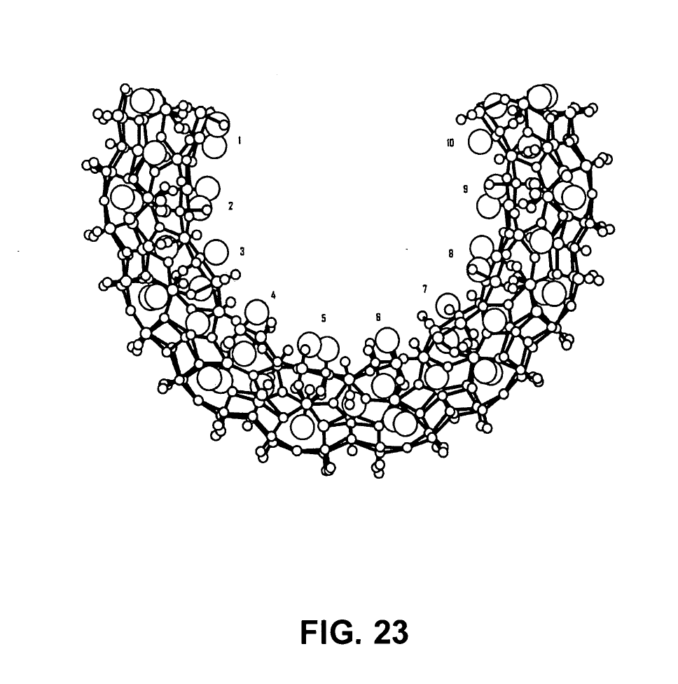

FIG. 23 shows an exemplary sub-molecular unit folded into a truncated ring conformation with functionalizable “addresses” shown by the ten (10) numbered, large single or double-spherical moieties on the inner portion of the ring. See U.S. Pat. No. 6,716,370 to Kendig.

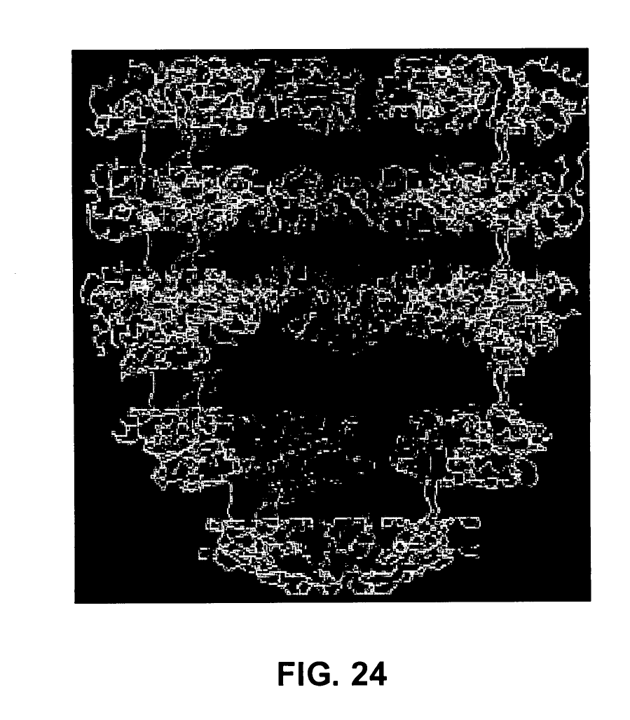

FIG. 24 shows an exemplary top-on view of five (5) sub-molecular units cross-linked into a supra-molecular structure, creating a catalytic cleft of progressively increasing cleft enclosure size—from bottom to top. Each unit can be orthogonally functionalized as described in FIGS. 21-23.

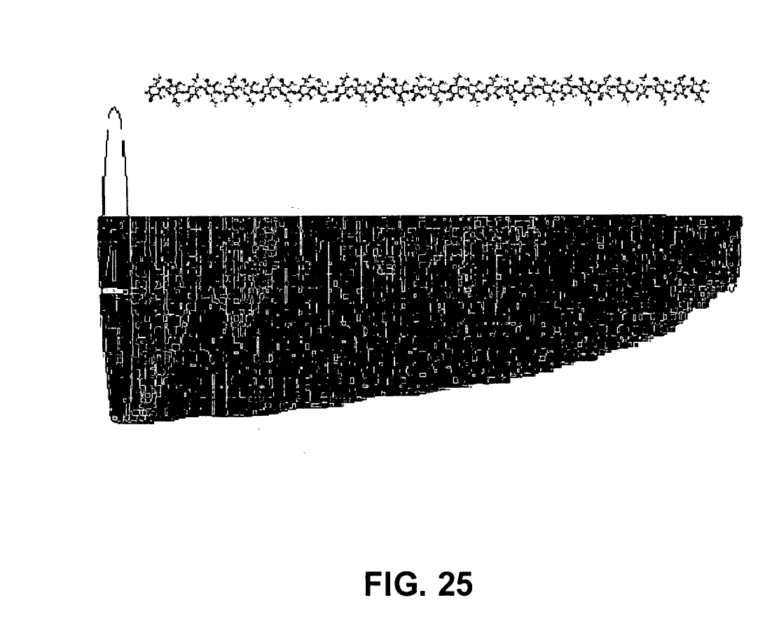

FIG. 25 shows an off angle side view of an exemplary idealized product of the catalytic cleft geometry, composed of a multiplicity of truncated ring shaped, and inner-surface functionalized, sub-molecular units that have been cross-linked into a supra-molecular structure of progressively increasing enclosure size—from right to left. Also shown is a circular ring “anchor,” or seed shape, on the extremity that is used as a polymerization guide for iterative addition of truncated ring units, to create and preserve the overall “cleft” conformation of the product. A short, 30 glucose monomer-long cellulose molecule is shown above for size comparison.

### INCORPORATION BY REFERENCE

Any and all references cited in the text of this patent application, including any U.S. or foreign patents or published patent applications, International patent applications, as well as, any non-patent literature reference are hereby expressly incorporated by reference.

The present invention has been described as an artificial enzyme. It will be understood that the above description is merely illustrative of the applications of the principles of the present invention, the scope of which is to be determined by the claims viewed in light of the specification. Other variants and modifications of the invention will be apparent to those of skill in the art.

We claim:

1-6. (canceled)

7. An artificial enzyme comprising (a) an organic polymer and (b) an active site displaying biocatalytic functionality.

8. An artificial enzyme of claim 7, wherein the organic polymer presents specific catalytically active atoms optimally for reactions so as to create an active site.

9. An artificial enzyme as in claim 7, wherein the active site comprises an assembly of monomer units in a predetermined three-dimensional orientation.

10. An artificial enzyme as in claim 7, wherein the organic polymer comprises a plurality of monomer units, and wherein the monomer units comprise one or more of plastic units, natural amino acids, artificial amino acids, molecules having electrophilic groups, molecules having nucleophilic groups, molecules contributing unique chemical functions not associated with the orthogonal functions inherent in natural amino acids.

11. An artificial enzyme as in claim 10, wherein the monomer units comprise one or more natural amino acids bearing at least one catalytically relevant side group, wherein the side group comprises one or more of amine, carboxyl, formamide, hydroxyl, mercaptyl, and saturated hydrocarbon.

12. An artificial enzyme as in claim 10, wherein the monomer units comprise one or more artificial amino acids comprised of alpha, beta, gamma, or other extended backbone amino acids.

13. An artificial enzyme as in claim 10, wherein the monomer units comprise a plurality of artificial amino acids which are functionalized at the nitrogen atom and are of varying backbone lengths.

14. An artificial enzyme as in claim 10, wherein the monomer units comprise one or more of: molecules having electrophilic groups, carboxylic acids, alkenes and alkynes.

15. An artificial enzyme as in claim 10, wherein the monomer units comprise one or more of: nucleophilic groups, primary amines, secondary amines, hydroxyl, mercaptyl and phosphate groups.

16. An artificial enzyme as in claim 7, wherein the organic polymer is the result of synthesis condensation, free radical propagation, or a dehydration chemical reaction.

17. An artificial enzyme as in claim 10, wherein the plastic monomer units are copolymerized to produce a polyurea, polyimide, polyurethane, polyacrylate, or polylactate.

18. An artificial enzyme as in claim 10, wherein the plurality of monomer units fold the organic polymer into a predetermined confirmation.

19. An artificial enzyme as in claim 7, wherein the biocatalytic functionality comprises one or more of keto-enol reactivity, ene-diol formation, Sn1 and Sn2 displacement, Diels-Alder reaction, general metathesis, a metallo-organic function, nitro aldol, Knoevenagel reaction, Morita-Baylis-Hillman reaction, Steglich rearrangement, 1,3-dipolar cycloaddition, Strecker synthesis, allylation, alkylation, halogenation or amination.

20. An artificial enzyme as in claim 7, wherein the organic polymer is the product of the copolymerization of one or more organic polymers, binding agents, or cross-linkers.

21. An artificial enzyme as in claim 7, wherein the artificial enzyme comprises one or more of the following geometric structures: dendrimers, spheres, fractal-patterned three-dimensional nets, block copolymers, layered copolymers, arrayed sheets, parallel sheets, and helices.

22. An artificial enzyme as in claim 7, wherein the artificial enzyme comprises a trough shape like that found in the catalytic regions of glycosyl hydrolases.

23. An artificial enzyme as in claim 10, wherein the monomer units comprise a plastic monomer that is orthogonally functionalized with chemical functions that contribute to catalysis.

24. An artificial enzyme as in claim 23, wherein the plastic monomer is orthogonally functionalized to accept an amino acid, DNA-based nucleotide, other catalytically contributive monomer, or combination thereof, that does not contribute to the overall shape or three-dimensional conformation of the artificial enzyme.

25. A method of producing an artificial enzyme, comprising copolymerizing catalytic monomers with noncatalytic monomers by one or more of the following: line copolymerization, backbone copolymerization, side group copolymerization, decoration copolymerization, mated copolymerization, classic copolymerization.

26. A method as in claim 25, wherein copolymerizing comprises heterogeneous copolymerization of plastic-based monomers with oligopeptides.

27. A method as in claim 25, wherein copolymerizing comprises heterogeneous copolymerization of plastic-based monomers with single strand DNA.
